(19) United States
(12) Patent Application Publication
(10) Pub. No.: US 2014/0090111 A1
(43) Pub. Date: Mar. 27, 2014

(54) Plant Hormone Biosensors

(71) Applicant: Carnegie Institution of Washington, Washington, DC (US)

(72) Inventors: Wolf B. Frommer, Washington, DC (US); Alexander M. Jones, Washington, DC (US)

(73) Assignee: Carnegie Institution of Washington, Washington, DC (US)

(21) Appl. No.: 14/019,062

(22) Filed: Sep. 5, 2013

Related U.S. Application Data
(60) Provisional application No. 61/697,155, filed on Sep. 5, 2012.

(51) Int. Cl. G01N 33/74; G01N 33/58

(52) U.S. Cl. CPC G01N 33/74; G01N 33/582
USPC 800/298; 530/350; 536/23.4; 435/320.1; 435/69.7; 436/501; 435/7.1; 424/9.6; 435/252.3; 435/252.33; 435/252.31; 435/252.34; 435/252.35; 435/419; 435/367; 435/361; 435/365; 435/369; 435/357; 435/254.2; 435/254.21; 435/254.23; 435/416; 435/415; 435/411; 435/417; 435/412

(57) ABSTRACT

The invention provides fusion proteins comprising at least two fluorescent proteins, with the fluorescent proteins emitting different wavelengths of light from one another, at least one plant hormone binding domain that changes three-dimensional conformation upon specifically binding to a plant hormone, and two linker peptides, with the first linker linking the first fluorescent protein to the N-terminus of the plant hormone binding domain and the second linker linking the second fluorescent protein to the C-terminus of the plant hormone binding domain. The invention also provides for methods of using the fusion proteins of the present invention and nucleic acids encoding the fusion proteins.

### STATEMENT REGARDING FEDERALLY SPONSORED RESEARCH OR DEVELOPMENT

Part of the work performed during development of this invention utilized U.S. Government funds through National Science Foundation Grant No. 1045185. The U.S. Government has certain rights in this invention.

### CROSS-REFERENCE TO RELATED APPLICATIONS

N/A

### SEQUENCE LISTING INFORMATION

N/A

### BACKGROUND OF THE INVENTION

1. Field of the Invention

The present invention is directed towards fusion proteins that bind plant hormones.

2. Background of the Invention

Over ten phytohormones are now known today, however the study of hormone function is complicated by chemical diversity and low abundance. Due to their transient and local action, e.g at the cell surface or in specific compartments of the cell, as well as in specific cells only, as such, accurate and facile methods for measuring plant hormones in living tissue are rare, despite their significance. Multiplexed gas chromatography mass spectrometry (GC-MS) protocols have improved ease of analysis, sensitivity and accuracy for hormone measurements. A major limitation is that classical techniques are not applicable to intact living tissues and have limited spatial and temporal resolution. An alternative approach for such analysis has been the engineering of promoter-reporter constructs sensitive to hormone concentration changes. These constructs have been useful for investigating auxin and cytokinin levels, but is limited by the indirect nature of the reporters and again has limited spatial and temporal resolution. For example, transcriptional reporters such as the auxin reporter DR5::GFP cannot reveal sub-cellular compartmentalization, and cannot accurately reflect the highly transient events, as they often respond to signals other than the target hormone, and they miss important hormone dynamics that fall below threshold concentrations for activation. For example, the most advanced auxin sensor DU-Venus reports auxin levels as a consequence of the degradation of the DII-domain, is thus indirect and has a negative output. Report kinetics are dominated by the degradation kinetics. Despite the progress in the case of auxin, new analytic tools are needed to provide the next level of resolution in hormone biology. Even in the case of auxin, measurements using known biosensors are insufficient for providing a critical validation and elaboration of the developmental models currently available. For most other plant hormones, biosensors could provide the first high-resolution measurements leading to entirely new systems level understanding.

Accordingly, there is a need for additional biosensors that can measure the presence or absence of specific plant hormones in living systems and in experimental settings.

### SUMMARY OF THE INVENTION

The invention provides fusion proteins comprising at least two fluorescent proteins, with the fluorescent proteins emitting different wavelengths of light from one another, at least one plant hormone binding domain that changes three-dimensional conformation upon specifically binding to a plant hormone, and two linker peptides, with the first linker linking the first fluorescent protein to the N-terminus of the plant hormone binding domain and the second linker linking the second fluorescent protein to the C-terminus of the plant hormone binding domain.

The invention also provides for methods of measuring plant hormones in a sample, comprising contacting the sample with a fusion protein of the present invention.

The present invention also provides for nucleic acids encoding the fusion proteins of the present invention.

### BRIEF DESCRIPTION OF THE DRAWINGS

FIG. 1 depicts a 96-well array of SMS designs that shows a subset of cloned hormone binding domains. This 96-well array of SMS designs shows a subset of cloned hormone binding domains. This set has two subdomains, listed in order. The two subdomains are linked by one of five linkers of different size and molecular properties (L10, L45, L60, L64 or L111). Thus, this array represents 480 Entry clones. Each entry clone can be combined with the destination vector library to yield a diversity of possible SMS designs that can then be screened in high-throughput.

FIG. 2 depicts two examples vectors that could be used in generating the fusion proteins of the present invention.

FIG. 3 depicts the fluorescence emission curves of ABA sensors. Signaling molecule sensors expressed in yeast were tested for response to analytes in cleared cell lysates or after purification. These ABA sensors were all FRET based biosensors responsive to samples containing ABA. SMS33 and SMS31 are single domain sensors with PYL10 and PYL8 as the ABA binding domains, respectively. SMSX designs have two sub-domains, i.e., SMSX107 contains PYL8 and ABI1, and SMSX31 contains PYL5 and HAB1. The SMSX designs depicted also have linker domain (L10, L45, L60, L64 or L111). All of the biosensors shown were expressed from pDRFLIP39, which has eCFP as the FRET donor and Venus as the FRET acceptor.

FIG. 4 depicts arrays of fluorescence emission curves from an ABA biosensor optimization screen. Left: Screening of 24 two-domain ABA biosensor designs with the L111 linker and the fluorescent proteins of pDRFLIP39 (sAFP-sCFPt). Light gray is mock treated, dark gray is ABA treated. The combination of an ABI1 truncation as the first domain and either PYR1 or PYL1 as the second domain (SMSX109 and SMSX110) resulted in a sensor with large ratio change. Right: Two sensors from the array at left were selected to be tested with the full set of five linkers and a set of eight pDRFLIPs. The pDRFLIP38 based sensors have an improved variant of CFP termed Cerulean.

FIG. 5 depicts the sensor response of two different ABA sensors. Both were responsive to ABA.

FIG. 6 depicts the concentration dependence and the selectiveness of an abscisic acid (ABA)-sensitive fusion protein of the present invention to different plant hormones and other chemicals: NaOH (Sodium Hydroxide), JA (jasmonic acid), GA (giberrelic acid), IAA (auxin), abscisic acid (ABA) and Kinetin (Kin). The signal intensity was concentration dependent. A: Response of SMSX110L111.DR39 to samples containing different concentrations of ABA. B: Response of the same sensor to samples containing ABA or various control compounds.

FIG. 7 depicts the ribbon structure of an ABA-sensitive fusion protein of the present invention.

FIG. 8 depicts ABA response of ABA biosensors with mutations in ABA binding sites and PYL-homodimerization sites. Mutations that reduced the homo-dimerization of ABA receptors, e.g., PYR1 and PYL1, result in higher affinity binding of ABA. The H87P mutation of the PYL1 subdomain in SMSX110L45.DR38 resulted in a higher-affinity ABA biosensor. Mutations that reduced binding of the PP2C co-receptor resulted in lower affinity binding of ABA by the receptors. The W300A mutation of the ABI1 subdomain in SMSX110L45.DR38 resulted in a lower-affinity ABA biosensor. Mutations that abolished the binding of the ABA receptors destroyed ABA sensing. The K86A mutation of the PYL1 subdomain in SMSX110L45.DR38 resulted in a non-binding ABA biosensor, and this construct can serve as negative control for various applications.

FIG. 9 depicts the fluorescence emission curves of a GA biosensor in response to GA. Also depicted are the GA responses of biosensor mutant variants. A: GA response of SMSX39L10.DR43 (RGA2 and GID1C sub-domains, L10 linker, Venus and Cerulean fluorescent proteins). B: Mutations of a GA binding residue of GID1C(S114A) resulted in a reduced GA affinity biosensor. Mutations that abolished (RGA2 DELLA domain deletion) or rendered constitutive (GID1C P98A) GID interaction with DELLA result in a GA non-responsive biosensor that serve as useful controls.

### DETAILED DESCRIPTION OF THE INVENTION

The invention provides fusion proteins comprising at least two fluorescent proteins, with the fluorescent proteins emitting different wavelengths of light from one another, at least one plant hormone binding domain that changes three-dimensional conformation upon specifically binding to a plant hormone, and two linker peptides, with the first linker linking the first fluorescent protein to the N-terminus of the plant hormone binding domain and the second linker linking the second fluorescent protein to the C-terminus of the plant hormone binding domain. The fusion proteins of the present invention may or may not be isolated.

The terms “peptide,” “polypeptide” and “protein” are used interchangeably herein. As used herein, an “isolated polypeptide” is intended to mean a polypeptide that has been completely or partially removed from its native environment. For example, polypeptides that have been removed or purified from cells are considered isolated. In addition, recombinantly produced polypeptides molecules contained in host cells are considered isolated for the purposes of the present invention. Moreover, a peptide that is found in a cell, tissue or matrix in which it is not normally expressed or found is also considered as “isolated” for the purposes of the present invention. Similarly, polypeptides that have been synthesized are considered to be isolated polypeptides. “Purified,” on the other hand is well understood in the art and generally means that the peptides are substantially free of cellular material, cellular components, chemical precursors or other chemicals beyond, perhaps, buffer or solvent. “Substantially free” is not intended to mean that other components beyond the novel peptides are undetectable. The fusion proteins of the present invention may be isolated or purified.

As used herein, the term fusion protein is, generally speaking, used as it is in the art and means two peptide fragments covalently bonded to one another via a typical amine bond between the fusion partners, thus creating one contiguous amino acid chain.

The fusion proteins of the present invention comprise at least two different fluorescent proteins. As used herein, fluorescent proteins are determined to be “different” from one another by the wavelength of light that each protein emits. For example, two “different” fluorescent proteins as used herein will emit light at wavelengths that are different from one another. The invention also contemplates fusion proteins with more than two fluorescent proteins. For example, the fusion proteins of the present application may comprise three, four, five or even six fluorescent proteins, with at least two of the fluorescent proteins being different from one another. Of course, each of the two or more fluorescent proteins may be different from one another, as defined herein.

The term “fluorescent protein” is readily understood in the art and simply means a protein that emits fluorescence at a detectable wavelength. Examples of fluorescent proteins that are part of fusion proteins of the current invention include, but are not limited to, green fluorescent proteins (GFP, AcGFP, ZsGreen), red-shifted GFP (rs-GFP), red fluorescent proteins (RFP, including DsRed2, HcRed1, dsRed-Express, cherry, tdTomato), yellow fluorescent proteins (YFP, Zsyellow), cyan fluorescent proteins (CFP, AmCyan), a blue fluorescent protein (BFP), amertrine, citrine, cerulean, turquoise, VENUS, teal fluorescent protein (TFP), LOV (light, oxygen or voltage) domains, and the phycobiliproteins, as well as the enhanced versions and mutations of these proteins. Fluorescent proteins as well as enhanced versions thereof are well known in the art and are commercially available. For some fluorescent proteins, “enhancement” indicates optimization of emission by increasing the protein's brightness, creating proteins that have faster chromophore maturation and/or alteration of dimerization properties. These enhancements can be achieved through engineering mutations into the fluorescent proteins.

The fluorescent proteins, for example the phycobiliproteins, may be particularly useful for creating tandem dye labeled labeling reagents. In one embodiment of the current invention, therefore, the measurable signal of the fusion protein is actually a transfer of excitation energy (resonance energy transfer) from a donor molecule (e.g., a first fluorescent protein) to an acceptor molecule (e.g., a second fluorescent protein). In particular, the resonance energy transfer is in the form of fluorescence resonance energy transfer (FRET). When the fusion proteins of the present invention utilize FRET to measure or quantify analyte(s), one fluorescent protein of the fusion protein construct can be the donor, and the second fluorescent protein of the fusion protein construct can be the acceptor. The terms “donor” and “acceptor,” when used in relation to FRET, are readily understood in the art. Namely, a donor is the molecule that will absorb a photon of light and subsequently initiate energy transfer to the acceptor molecule. The acceptor molecule is the molecule that receives the energy transfer initiated by the donor and, in turn, emits a photon of light. The efficiency of FRET is dependent upon the distance between the two fluorescent partners and can be expressed mathematically by: E=R₀⁶/(R₀⁶+r⁶), where “E” is the efficiency of energy transfer, “r” is the distance (in Angstroms) between the fluorescent donor/acceptor pair and “R₀” is the Förster distance (in Angstroms). The Förster distance, which can be determined experimentally by readily available techniques in the art, is the distance at which FRET is half of the maximum possible FRET value for a given donor/acceptor pair. A particularly useful combination is the phycobiliproteins disclosed in U.S. Pat. Nos. 4,520,110; 4,859,582; 5,055,556, incorporated by reference, and the sulforhodamine fluorophores disclosed in U.S. Pat. No. 5,798,276, or the sulfonated cyanine fluorophores disclosed in U.S. Pat. Nos. 6,977,305 and 6,974,873; or the sulfonated xanthene derivatives disclosed in U.S. Pat. No. 6,130,101, incorporated by reference and those combinations disclosed in U.S. Pat. No. 4,542,104, incorporated by reference.

The fusion proteins also comprise at least one plant hormone binding domain. A “binding domain” is used herein as it is in the art. Namely, a binding domain is molecule that binds a target in a specific manner. Thus a “plant hormone binding domain” is a binding domain that specifically binds to one or more plant hormones. In one embodiment, the plant hormone binding domain is a protein. The binding domain may comprise an entire protein, such as a wild-type protein, or a portion thereof.

In one embodiment of the current invention, the binding domain comprises a single polypeptide or protein. In another embodiment, the binding domain comprises more than one subdomain, with each subdomain being a separate or distinct polypeptide or protein. As used herein in the context of subdomains, “a separate protein” does not necessarily mean that the proteins or polypeptides have distinct amino acid sequences. Instead, “a separate protein” for the purposes of distinguishing portions of the binding domain means that the each of the proteins of the subdomains are structurally independent and generally, but not necessarily, each have characteristics of small globular proteins. A “distinct protein,” on the other hand is used to mean proteins or polypeptides that have distinct amino acid sequences, with each protein of the subdomain having characteristics of small globular proteins. In specific embodiments, the fusion proteins of the present invention comprise one, two, three, four, five or six subdomains.

In one embodiment, when the plant hormone binding domain comprises more than one subdomain, the subdomains are linked together without a linker peptide such that the C-terminus of one subdomain is linked via a typical amine bond to the N-terminus of the another subdomain. In another embodiment, when the plant hormone binding domain comprises more than one subdomain, the subdomains are linked together with a linker peptide, i.e., “a subdomain linker peptide.” As used herein, a subdomain linker peptide is a used to mean a polypeptide typically ranging from about 1 to about 120 amino acids in length that is designed to facilitate the functional connection of two subdomains into a linked binding domain. To be clear, a single amino acid can be considered a subdomain linker peptide for the purposes of the present invention. In specific embodiments, the subdomain linker peptide comprises or in the alternative consists of amino acids numbering 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 residues in length. Of course, the subdomain linker peptides used in the fusion proteins of the present invention may comprise or in the alternative consist of amino acids numbering more than 120 residue in length. The length of the subdomain linker peptide, if present, may not be critical to the function of the fusion protein, provided that the subdomain linker peptide permits a functional connection between the subdomains.

The term “functional connection” in the context of a linker peptide indicates a connection that facilitates folding of the polypeptides of each subdomain into a three dimensional structure that allows the linked fusion polypeptide to mimic some or all of the functional aspects or biological activities of the domain from which its subdomain constituents are derived. For example, in the case of an abscisic acid (ABA) binding domain, the linker may be used to create a single-chain fusion of a multi-subdomain protein to achieve the desired biological activity of binding ABA or to achieve a three dimensional structure that mimics the structure of each of the native subdomains. The term functional connection also indicates that the linked subdomains possess at least a minimal degree of stability, flexibility and/or tension that would be required for the binding domain to function as desired.

In one embodiment of the present invention, the subdomain linker peptides comprise or consist of the same amino acid sequence. In another embodiment, the amino acid sequences of the subdomain linker peptides are different from one another.

In specific embodiment, the subdomain linker peptide comprises or consists of a peptide with the amino acid sequence of SEQ ID NO:1, SEQ ID NO:2, SEQ ID NO:3, SEQ ID NO:4 or SEQ ID NO:5. In additional embodiments, the amino acid sequence of one subdomain linker peptide comprises the amino acid sequence of SEQ ID NO:1, SEQ ID NO:2, SEQ ID NO:3, SEQ ID NO:4 or SEQ ID NO:5 and the amino acid sequence of the other subdomain linker peptide comprises the amino acid sequence of SEQ ID NO:1, SEQ ID NO:2, SEQ ID NO:3, SEQ ID NO:4 or SEQ ID NO:5.

The fusion proteins of the present invention comprise at least one plant hormone binding domain, with the binding domain comprising at least one subdomain. As used a “plant hormone binding domain” is a receptor or portion thereof that specifically binds to one or more plant hormones. The term plant hormone binding domain” is also used to mean an effector molecule that directly binds to a hormone receptor once the plant hormone is bound to the hormone receptor. An example of a plant hormone receptor would include the transport inhibitor response 1 (TIR1) and TIR1-like receptors (AFBs), and an effector molecule that binds to an auxin-bound TIR1 receptor would include but would not be limited to IAA1, IAA2, IAA3, IAA4, IAA5, IAA6, IAA7, IAA8, IAA9, IAA10, IAA11, IAA12, IAA13, IAA14, IAA15, IAA16, IAA17, IAA18, IAA19, IAA26, IAA27, IAA28, IAA29 and IAA31 proteins or portions thereof. Continuing the example, all of the listed TIR, TIR1-like molecules and IAA molecules would be considered an auxin-binding domain for the purposes of the present invention.

A “plant hormone,” in turn, is used to indicate a plant-generated signaling molecule that normally affects at least one aspect of plant development, including but not limited to, growth, seed development, flowering and root growth. One of skill in the art will readily understand the term plant hormone and what entities fall under the scope of this term. For example, plant hormones include but are not limited to, abscisic acid (ABA) or a derivative thereof, gibberellins (GA), auxins (IAA), ethylene, cytokinins (CK), brassinosteroids (BR), jasmonates (JA), salicylic acid (SA), strigolactones (SL). In select embodiments, the fusion proteins of the present invention comprise a plant hormone binding domain that binds abscisic acid (ABA), gibberellins (GA), auxins (IAA) and/or jasmonates (JA).

ABA binding domains include but are not limited to protein phosphate 2C (PP2C) (HAB1, ABI1, ABI2), Abscisic acid receptor PYR1 (PYR1), Abscisic acid receptor PYL1 (PYL1), Abscisic acid receptor PYL4 (PYL4), Abscisic acid receptor PYL5 (PYL5), Abscisic acid receptor PYL6 (PYL6), Abscisic acid receptor PYL7 (PYL7), Abscisic acid receptor PYL8 (PYL8), Abscisic acid receptor PYL9 (PYL9) and Abscisic acid receptor PYL10 (PYL10). In several embodiments, the plant ABA binding domain of the fusion proteins of the present invention comprises one plant ABA binding domain comprising a binding domain, the binding domain being PP2C, PYR1, PYL1, PYL4, PYL5, PYL8, PYL9 or portions thereof. In another embodiment, the ABA binding domain of the fusion proteins of the present invention comprise a plurality of at least two subdomains, with the each of the subdomains being HAB1, ABI1, ABI2, PYR1, PYL1, PYL4, PYL5, PYL6, PYL7, PYL8, PYL9 and PYL10 or portions thereof. The fusion proteins may comprise any combination of the listed subdomains. The fusion protein comprising at least a plurality of subdomains may also comprise more than one copy of the same subdomain, for example two PYL9 proteins.

In more specific embodiments, the fusion protein comprises an ABA binding domain, the ABA binding domain comprising two subdomains with the first subdomain selected from the group consisting of ABI1 and HAB1, and the second subdomain selected from the group consisting of PYR1, PYL1, PYL4, PYL5, PYL6, PYL7, PYL8, PYL9 and PYL10.

IAA binding domains include but are not limited to transport inhibitor response 1 (TIR1), TIR1-like receptors (AFB), and an effector such as but not limited to auxin responsive protein 1 (IAA1), IAA2, IAA3, IAA4, IAA5, IAA6, IAA7, IAA8, IAA9, IAA10, IAA11, IAA12, IAA13, IAA14, IAA15, IAA16, IAA17, IAA18, IAA19, IAA26, IAA27, IAA28, IAA29, IAA31, or proteins or portions thereof. In several embodiments, the plant IAA binding domain of the fusion proteins of the present invention comprises one plant IAA binding domain comprising a binding domain, the binding domain being TIR1, AFB, IAA1, IAA3, IAA7, IAA8, IAA9, IAA12, IAA17, IAA28, or proteins or portions thereof. In another embodiment, the IAA binding domain of the fusion proteins of the present invention comprise a plurality of at least two subdomains, with the each of the subdomains being TIR1, AFB, IAA1, IAA3, IAA7, IAA8, IAA9, IAA12, IAA17, IAA28 or portions thereof. The fusion proteins may comprise any combination of the listed subdomains. The fusion protein comprising at least a plurality of subdomains may also comprise more than one copy of the same subdomain, for example two IAA1 proteins.

In more specific embodiments, the fusion protein comprises an IAA binding domain, the IAA binding domain comprising two subdomains with the first subdomain being TIR1, and the second subdomain selected from the group consisting of IAA1, IAA3, IAA7, IAA8, IAA9, IAA12, IAA17 and IAA28.

GA binding domains include but are not limited to gibberellin receptor 1A (GID1A), GID1B, GID1C, GID1D, DELLA RGA1 protein (RGA1), RGA2 and RGL1 and proteins or portions thereof. In several embodiments, the plant GA binding domain of the fusion proteins of the present invention comprises one plant GA binding domain comprising a binding domain, the binding domain being GID1A, GID1B, GIB1C, RGA1, RGA2, RGL1 or proteins or portions thereof. In another embodiment, the GA binding domain of the fusion proteins of the present invention comprise a plurality of at least two subdomains, with the each of the subdomains being GID1A, GID1B, GID1C, RGA1, RGA2, RGL1 or proteins or portions thereof. The fusion proteins may comprise any combination of the listed subdomains. The fusion protein comprising at least a plurality of subdomains may also comprise more than one copy of the same subdomain, for example two GID1 proteins.

In more specific embodiments, the fusion protein comprises a GA binding domain, the GA binding domain comprising two subdomains with the first subdomain selected from the group consisting of RGA1 and RGA2, and the second subdomain selected from the group consisting of GID1A, GID1B and GID1C.

JA binding domains include but are not limited to coronatine insensitive protein 1 (COI1), jasmonate-zim-domain protein 1 (JAZ1 and also known as protein TIFY10A (TIFY10A)), JAZZ, JAZ3, JAZ4, JAZ5, JAZ6, JAZ7, JAZ8, JAZ9, JAZ10, JAZ11 and JAZ12 or portions thereof. In several embodiments, the plant JA binding domain of the fusion proteins of the present invention comprises one plant JA binding domain comprising a binding domain, the binding domain being COI1, JAZ1, JAZ3, JAZ6, JAZ9 or proteins or portions thereof. In another embodiment, the JA binding domain of the fusion proteins of the present invention comprise a plurality of at least two subdomains, with the each of the subdomains being COI1, JAZ1, JAZ3, JAZ6, JAZ9 or proteins or portions thereof. The fusion proteins may comprise any combination of the listed subdomains. The fusion protein comprising at least a plurality of subdomains may also comprise more than one copy of the same subdomain, for example two COI1 proteins.

In more specific embodiments, the fusion protein comprises a JA binding domain, the JA binding domain comprising two subdomains with the first subdomain being COI1, and the second subdomain selected from the group consisting of JAZ1, JAZ3, JAZ6 and JAZ9.

The plant hormone binding domains can be from any plant source and the invention is not limited by the source of the binding domain, i.e., the invention is not limited to the plant species from which the binding domain normally occurs or is obtained. Examples of sources from which the plant hormone binding domains may be derived include but are not limited to monocotyledonous plants that include, for example, Lolium, Zea, Triticum, Sorghum, Triticale, Saccharum, Bromus, Oryzae, Avena, Hordeum, Secale and Setaria. Other sources from which the plant hormone binding domains may be derived include but are not limited to maize, wheat, barley, rye, rice, oat, sorghum and millet. Additional sources from which the plant hormone binding domains may be derived include but are not limited to dicotyledenous plants that include but are not limited to Fabaceae, Solanum, Brassicaceae, especially potatoes, beans, cabbages, forest trees, roses, clematis, oilseed rape, sunflower, chrysanthemum, poinsettia, arabidopsis, tobacco, tomato, and antirrhinum (snapdragon), soybean, canola, sunflower and even basal land plant species, (the moss Physcomitrella patens). Additional sources also include gymnosperms.

It is understood that the invention is not limited to plant hormone binding domains from the plant species listed herein, and that the invention encompasses proteins encoded by orthologous of genes in other species. For example, it is understood that fusion proteins comprising the PYL1 protein in Arabidopsis can be applied to the PYL1 protein encoded by the orthologous gene in another species. As used herein, orthologous genes are genes from different species that perform the same or similar function and are believed to descend from a common ancestral gene. Proteins from orthologous genes, in turn, are the proteins encoded by the orthologs. As such the term “ortholog” may be to refer to a gene or a protein. Often, proteins encoded by orthologous genes have similar or nearly identical amino acid sequence identities to one another, and the orthologous genes themselves have similar nucleotide sequences, particularly when the redundancy of the genetic code is taken into account. Thus, by way of example, the ortholog of the PYL1 receptor Arabidopsis would be a PYL1 receptor in another species of plant, regardless of the amino acid sequence of the two proteins.

In another aspect, the invention provides deletion variants wherein one or more amino acid residues in the plant hormone binding domain or one or more fluorescent protein(s) are removed. Deletions can be effected at one or both termini of the plant hormone binding domain or one or more fluorescent protein(s), or with removal of one or more non-terminal amino acid residues of the plant hormone binding domain or one or more fluorescent protein(s).

The fusion proteins of the present invention may also comprise substitution variants of a plant hormone binding domain or subdomain. Substitution variants include those polypeptides wherein one or more amino acid residues of the plant hormone binding domains are removed and replaced with alternative residues. In general, the substitutions are conservative in nature. Conservative substitutions for this purpose may be defined as set out in the tables below. Amino acids can be classified according to physical properties and contribution to secondary and tertiary protein structure. A conservative substitution is recognized in the art as a substitution of one amino acid for another amino acid that has similar properties. Exemplary conservative substitutions are set out in below.

TABLE I
Conservative Substitutions

| Side Chain Characteristic | Amino Acid |
|---|---|
| Aliphatic | |
| Non-polar | Gly, Ala, Pro, Iso, Leu, Val |
| Polar-uncharged | Cys, Ser, Thr, Met, Asn, Gln |
| Polar-charged | Asp, Glu, Lys, Arg |
| Aromatic | His, Phe, Trp, Tyr |
| Other | Asn, Gln, Asp, Glu |

Alternatively, conservative amino acids can be grouped as described in Lehninger (1975) Biochemistry, Second Edition; Worth Publishers, pp. 71-77, as set forth below.

TABLE II
Conservative Substitutions

| Side Chain Characteristic | Amino Acid |
|---|---|
| Non-polar (hydrophobic) | |
| Aliphatic: | Ala, Leu, Iso, Val, Pro |
| Aromatic: | Phe, Trp |
| Sulfur-containing: | Met |
| Borderline: | Gly |
| Uncharged-polar | |
| Hydroxyl: | Ser, Thr, Tyr |
| Amides: | Asn, Gln |
| Sulfhydryl: | Cys |
| Borderline: | Gly |
| Positively Charged (Basic): | Lys, Arg, His |
| Negatively Charged (Acidic) | Asp, Glu |

And still other alternative, exemplary conservative substitutions are set out below.

TABLE III
Conservative Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

In one embodiment, the plant hormone binding domain is linked to the fluorescent proteins without a linker peptide such that the N-terminus of the hormone binding domain is linked via a typical amine bond to the C-terminus of one fluorescent protein, and the C-terminus of the hormone binding domain is linked via a typical amine bond to the N-terminus of another fluorescent protein. In another embodiment, the plant hormone binding domain is linked to the fluorescent proteins with a linker peptide, i.e., “a fluorescent protein linker peptide.” In yet another embodiment, the plant hormone binding domain is linked to one of the fluorescent proteins with a linker peptide and is linked to the other fluorescent protein without a linker peptide. In the embodiment when only one fluorescent protein linker peptide is used, either the N-terminus or the C-terminus of the hormone binding domain can be the location of the fluorescent protein linker peptide. As used herein, a fluorescent protein linker peptide is used to mean a polypeptide typically ranging from about 1 to about 50 amino acids in length that is designed to facilitate the functional connection of a fluorescent protein to the hormone binding domain. To be clear, a single amino acid can be considered a fluorescent protein linker peptide for the purposes of the present invention. In specific embodiments, the fluorescent protein linker peptide comprises or in the alternative consists of amino acids numbering 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 residues in length. Of course, the fluorescent protein linker peptides used in the fusion proteins of the present invention may comprise or in the alternative consist of amino acids numbering more that 50 residue in length. The length of the fluorescent protein linker peptide, if present, may not be critical to the function of the fusion protein, provided that the fluorescent protein linker peptide permits a functional connection between the fluorescent protein and the hormone binding domain.

The term “functional connection” in the context of a linker peptide indicates a connection that facilitates folding of the hormone binding domain and the fluorescent proteins into a three dimensional structure that allows each of the portions of the fusion protein to mimic some or all of the functional aspects or biological activities of the hormone binding domain and fluorescent proteins.

In one embodiment of the present invention, the fluorescent protein linker peptides comprise or consist of the same amino acid sequence. In another embodiment, the amino acid sequences of the fluorescent protein linker peptides are different from one another.

In specific embodiment, the protein linker peptides comprise or consists of a peptide with the amino acid sequence of SEQ ID NO:6 or SEQ ID NO:7. In another embodiment, the amino acid sequence of one fluorescent protein linker peptide comprises the amino acid sequence of SEQ ID NO:6 and the amino acid sequence of the other fluorescent protein linker peptide comprises the amino acid sequence of SEQ ID NO:7.

In one embodiment of the present invention, the subdomain linker peptides comprise or consist of the same amino acid sequence as the fluorescent protein linker peptides. In another embodiment, the amino acid sequences of the subdomain linker peptides are different from the fluorescent protein linker peptides.

The fusion proteins of the present invention may or may not contain additional elements that, for example, may include but are not limited to regions to facilitate purification. For example, “histidine tags” (“his tags”) or “lysine tags” may be appended to the fusion protein. Examples of histidine tags include, but are not limited to hexaH, heptaH and hexaHN. Examples of lysine tags include, but are not limited to pentaL, heptaL and FLAG. Such regions may be removed prior to final preparation of the fusion protein. Other examples of a second fusion peptide include, but are not limited to, glutathione S-transferase (GST) and alkaline phosphatase (AP).

The addition of peptide moieties to fusion proteins, whether to engender secretion or excretion, to improve stability and to facilitate purification or translocation, among others, is a familiar and routine technique in the art and may include modifying amino acids at the terminus to accommodate the tags. For example the N-terminus amino acid may be modified to, for example, arginine and/or serine to accommodate a tag. Of course, the amino acid residues of the C-terminus may also be modified to accommodate tags. One particularly useful fusion protein comprises a heterologous region from immunoglobulin that can be used solubilize proteins.

Other types of fusion proteins provided by the present invention include but are not limited to, fusions with secretion signals and other heterologous functional regions. Thus, for instance, a region of additional amino acids, particularly charged amino acids, may be added to the N-terminus of the protein to improve stability and persistence in the host cell, during purification or during subsequent handling and storage.

The fusion proteins of the current invention can be recovered and purified from recombinant cell cultures by well-known methods including, but not limited to, ammonium sulfate or ethanol precipitation, acid extraction, anion or cation exchange chromatography, phosphocellulose chromatography, hydrophobic interaction chromatography, affinity chromatography, e.g., immobilized metal affinity chromatography (IMAC), hydroxylapatite chromatography and lectin chromatography. High performance liquid chromatography (“HPLC”) may also be employed for purification. Well-known techniques for refolding protein may be employed to regenerate active conformation when the fusion protein is denatured during isolation and/or purification.

Fusion proteins of the present invention include, but are not limited to, products of chemical synthetic procedures and products produced by recombinant techniques from a prokaryotic or eukaryotic host, including, for example, bacterial, yeast, higher plant, insect and mammalian cells. Depending upon the host employed in a recombinant production procedure, the fusion proteins of the present invention may be glycosylated or may be non-glycosylated. In addition, fusion proteins of the invention may also include an initial modified methionine residue, in some cases as a result of host-mediated processes.

As used herein, the terms “correspond(s) to” and “corresponding to,” as they relate to sequence alignment, are intended to mean enumerated positions within a reference protein, e.g., wild-type ABI-1, and those positions in a modified ABI-1 that align with the positions on the reference protein. Thus, when the amino acid sequence of a subject protein is aligned with the amino acid sequence of a reference protein, the amino acids in the subject sequence that “correspond to” certain enumerated positions of the reference sequence are those that align with these positions of the reference sequence, but are not necessarily in these exact numerical positions of the reference sequence. Methods for aligning sequences for determining corresponding amino acids between sequences are described herein.

The invention also relates to isolated nucleic acids and to constructs comprising these nucleic acids. The nucleic acids of the invention can be DNA or RNA, for example, mRNA. The nucleic acid molecules can be double-stranded or single-stranded; single stranded RNA or DNA can be the coding, or sense, strand or the non-coding, or antisense, strand. In particular, the nucleic acids may encode any fusion proteins of the invention. For example, the nucleic acids of the invention include polynucleotide sequences that encode the fusion proteins that contain or comprise glutathione-S-transferase (GST) fusion protein, poly-histidine (e.g., His₆), poly-HN, poly-lysine, etc. If desired, the nucleotide sequence of the isolated nucleic acid can include additional non-coding sequences such as non-coding 3′ and 5′ sequences (including regulatory sequences, for example).

The present invention also comprises vectors containing the nucleic acids encoding the fusion proteins of the present invention. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. An expression vector is one into which a desired DNA sequence may be inserted by restriction and ligation such that it is operably joined to regulatory sequences and may be expressed as an RNA transcript. Vectors may further contain one or more marker sequences suitable for use in the identification and selection of cells which have been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Examples of vectors include but are not limited to those capable of autonomous replication and expression of the structural gene products present in the DNA segments to which they are operably joined.

In certain respects, the vectors to be used are those for expression of polynucleotides and proteins of the present invention. Generally, such vectors comprise cis-acting control regions effective for expression in a host operatively linked to the polynucleotide to be expressed. Appropriate trans-acting factors are supplied by the host, supplied by a complementing vector or supplied by the vector itself upon introduction into the host.

A great variety of expression vectors can be used to express the proteins of the invention. Such vectors include chromosomal, episomal and virus-derived vectors, e.g., vectors derived from bacterial plasmids, from bacteriophage, from yeast episomes, from yeast chromosomal elements, from viruses such as adeno-associated virus, lentivirus, baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. All may be used for expression in accordance with this aspect of the present invention. Generally, any vector suitable to maintain, propagate or the fusion proteins in a host may be used for expression in this regard.

The DNA sequence in the expression vector is operatively linked to appropriate expression control sequence(s) including, for instance, a promoter to direct mRNA transcription. Representatives of such promoters include, but are not limited to, the phage lambda PL promoter, the E. coli lac, trp and tac promoters, HIV promoters, the SV40 early and late promoters and promoters of retroviral LTRs, to name just a few of the well-known promoters. In general, expression constructs will contain sites for transcription, initiation and termination and, in the transcribed region, a ribosome binding site for translation. The coding portion of the mature transcripts expressed by the constructs will include a translation initiating AUG at the beginning and a termination codon (UAA, UGA or UAG) appropriately positioned at the end of the polypeptide to be translated.

In addition, the constructs may contain control regions that regulate, as well as engender expression. Generally, such regions will operate by controlling transcription, such as repressor binding sites and enhancers, among others.

Vectors for propagation and expression generally will include selectable markers. Such markers also may be suitable for amplification or the vectors may contain additional markers for this purpose. In this regard, the expression vectors may contain one or more selectable marker genes to provide a phenotypic trait for selection of transformed host cells. Preferred markers include dihydrofolate reductase or neomycin resistance for eukaryotic cell culture, and tetracycline, kanamycin or ampicillin resistance genes for culturing E. coli and other bacteria.

Examples of vectors that may be useful for fusion proteins include, but are not limited to, pPZP, pZPuFLIPs, pCAMBIA, and pRT to name a few.

Examples of vectors for expression in yeast S. cerevisiae include pDRFLIP,s, pDR196, pYepSecl (Baldari (1987) EMBO J. 6, 229-234), pMFa (Kurjan (1982) Cell 30, 933-943), pJRY88 (Schultz (1987) Gene 54, 115-123), pYES2 (Invitrogen) and picZ (Invitrogen).

Alternatively, the fusion proteins can be expressed in insect cells using baculovirus expression vectors. Baculovirus vectors available for expression of proteins in cultured insect cells (e.g., SF9 cells) include the pAc series (Smith (1983) Mol. Cell. Biol. 3, 2156 2165) and the pVL series (Lucklow (1989) Virology 170, 31-39).

The nucleic acid molecules of the invention can be “isolated.” As used herein, an “isolated” nucleic acid molecule or nucleotide sequence is intended to mean a nucleic acid molecule or nucleotide sequence that is not flanked by nucleotide sequences normally flanking the gene or nucleotide sequence (as in genomic sequences) and/or has been completely or partially removed from its native environment (e.g., a cell, tissue). For example, nucleic acid molecules that have been removed or purified from cells are considered isolated. In some instances, the isolated material will form part of a composition (for example, a crude extract containing other substances), buffer system or reagent mix. In other circumstances, the material may be purified to near homogeneity, for example as determined by PAGE or column chromatography such as HPLC. Thus, an isolated nucleic acid molecule or nucleotide sequence can includes a nucleic acid molecule or nucleotide sequence which is synthesized chemically, using recombinant DNA technology or using any other suitable method. To be clear, a nucleic acid contained in a vector would be included in the definition of “isolated” as used herein. Also, isolated nucleotide sequences include recombinant nucleic acid molecules (e.g., DNA, RNA) in heterologous organisms, as well as partially or substantially purified nucleic acids in solution. “Purified,” on the other hand is well understood in the art and generally means that the nucleic acid molecules are substantially free of cellular material, cellular components, chemical precursors or other chemicals beyond, perhaps, buffer or solvent. “Substantially free” is not intended to mean that other components beyond the novel nucleic acid molecules are undetectable. The nucleic acid molecules of the present invention may be isolated or purified. Both in vivo and in vitro RNA transcripts of a DNA molecule of the present invention are also encompassed by “isolated” nucleotide sequences.

The invention also provides nucleic acid molecules that hybridize under high stringency hybridization conditions, such as for selective hybridization, to the nucleotide sequences described herein (e.g., nucleic acid molecules which specifically hybridize to a nucleotide sequence encoding fusion proteins described herein and encode a plant hormone binding domain and/or one or more fluorescent proteins). Hybridization probes include synthetic oligonucleotides which bind in a base-specific manner to a complementary strand of nucleic acid.

Such nucleic acid molecules can be detected and/or isolated by specific hybridization e.g., under high stringency conditions. “Stringency conditions” for hybridization is a term of art that refers to the incubation and wash conditions, e.g., conditions of temperature and buffer concentration, which permit hybridization of a particular nucleic acid to a second nucleic acid; the first nucleic acid may be perfectly complementary, i.e., 100%, to the second, or the first and second may share some degree of complementarity, which is less than perfect, e.g., 60%, 75%, 85%, 95% or more. For example, certain high stringency conditions can be used which distinguish perfectly complementary nucleic acids from those of less complementarity.

“High stringency conditions”, “moderate stringency conditions” and “low stringency conditions” for nucleic acid hybridizations are explained in Current Protocols in Molecular Biology, John Wiley & Sons). The exact conditions which determine the stringency of hybridization depend not only on ionic strength, e.g., 0.2×SSC, 0.1×SSC of the wash buffers, temperature, e.g., room temperature, 42° C., 68° C., etc., and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions may be determined empirically.

By varying hybridization conditions from a level of stringency at which no hybridization occurs to a level at which hybridization is first observed, conditions which will allow a given sequence to hybridize with the most similar sequences in the sample can be determined. Exemplary conditions are described in Krause (1991) Methods in Enzymology, 200:546-556. Washing is the step in which conditions are usually set so as to determine a minimum level of complementarity of the hybrids. Generally, starting from the lowest temperature at which only homologous hybridization occurs, each degree (° C.) by which the final wash temperature is reduced, while holding SSC concentration constant, allows an increase by 1% in the maximum extent of mismatching among the sequences that hybridize. Generally, doubling the concentration of SSC results in an increase in Tm. Using these guidelines, the washing temperature can be determined empirically for high, moderate or low stringency, depending on the level of mismatch sought. Exemplary high stringency conditions include, but are not limited to, hybridization in 50% formamide, 1 M NaCl, 1% SDS at 37° C., and a wash in 0.1×SSC at 60° C. Example of progressively higher stringency conditions include, after hybridization, washing with 0.2×SSC and 0.1% SDS at about room temperature (low stringency conditions); washing with 0.2×SSC, and 0.1% SDS at about 42° C. (moderate stringency conditions); and washing with 0.1×SSC at about 68° C. (high stringency conditions). Washing can be carried out using only one of these conditions, e.g., high stringency conditions, washing may encompass two or more of the stringency conditions in order of increasing stringency. Optimal conditions will vary, depending on the particular hybridization reaction involved, and can be determined empirically.

Equivalent conditions can be determined by varying one or more of the parameters given as an example, as known in the art, while maintaining a similar degree of identity or similarity between the target nucleic acid molecule and the primer or probe used. Hybridizable nucleotide sequences are useful as probes and primers for identification of organisms comprising a nucleic acid of the invention and/or to isolate a nucleic acid of the invention, for example. The term “primer” is used herein as it is in the art and refers to a single-stranded oligonucleotide which acts as a point of initiation of template-directed DNA synthesis under appropriate conditions in an appropriate buffer and at a suitable temperature. The appropriate length of a primer depends on the intended use of the primer, but typically ranges from about 15 to about 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

The present invention also relates to host cells containing the above-described constructs. The host cell can be a eukaryotic cell, such as a plant cell or yeast cell, or the host cell can be a prokaryotic cell, such as a bacterial cell. The host cell can be stably or transiently transfected with the construct. The polynucleotides may be introduced alone or with other polynucleotides. Such other polynucleotides may be introduced independently, co-introduced or introduced joined to the polynucleotides of the invention. As used herein, a “host cell” is a cell that normally does not contain any of the nucleotides of the present invention and contains at least one copy of the nucleotides of the present invention. Thus, a host cell as used herein can be a cell in a culture setting or the host cell can be in an organism setting where the host cell is part of an organism, organ or tissue.

If a prokaryotic expression vector is employed, then the appropriate host cell would be any prokaryotic cell capable of expressing the cloned sequences. Suitable prokaryotic cells include, but are not limited to, bacteria of the genera Escherichia, Bacillus, Pseudomonas, Staphylococcus, and Streptomyces.

If a eukaryotic expression vector is employed, then the appropriate host cell would be any eukaryotic cell capable of expressing the cloned sequence. In one embodiment, eukaryotic cells are the host cells. Eukaryotic host cells include, but are not limited to, insect cells, HeLa cells, Chinese hamster ovary cells (CHO cells), African green monkey kidney cells (COS cells), human 293 cells, and murine 3T3 fibroblasts.

In addition, a yeast cell may be employed as a host cell. Yeast cells include, but are not limited to, the genera Saccharomyces, Pichia and Kluveromyces. In one embodiment, the yeast hosts are S. cerevisiae or P. pastoris. Yeast vectors may contain an origin of replication sequence from a 2T yeast plasmid, an autonomously replication sequence (ARS), a promoter region, sequences for polyadenylation, sequences for transcription termination and a selectable marker gene. Shuttle vectors for replication in both yeast and E. coli are also included herein.

Introduction of a construct into the host cell can be affected by calcium phosphate transfection, DEAE-dextran mediated transfection, cationic lipid-mediated transfection, electroporation, transduction, infection or other methods.

Other examples of methods of introducing nucleic acids into host organisms take advantage TALEN technology to effectuate site-specific insertion of nucleic actions. TALENs are proteins that have been engineered to cleave nucleic acids at a specific site in the sequence. The cleavage sites of TALENs are extremely customizable and pairs of TALENs can be generated to create double-stranded breaks (DSBs) in nucleic acids at virtually any site in the nucleic acid. See Bogdanove and Voytas, Scienc, 333:1843-1846 (2011), which incorporated by reference herein

Transformants carrying the expression vectors are selected based on the above-mentioned selectable markers. Repeated clonal selection of the transformants using the selectable markers allows selection of stable cell lines expressing the fusion proteins constructs. Increased concentrations in the selection medium allows gene amplification and greater expression of the desired fusion proteins. The host cells, for example E. coli cells, containing the recombinant fusion proteins can be produced by cultivating the cells containing the fusion proteins expression vectors constitutively expressing the fusion proteins constructs.

The present invention also provides for transgenic plants or plant tissue comprising transgenic plant cells, i.e. comprising stably integrated into their genome, an above-described nucleic acid molecule, expression cassette or vector of the invention. The present invention also provides transgenic plants, plant cells or plant tissue obtainable by a method for their production as outlined below.

In one embodiment, the present invention provides a method for producing transgenic plants, plant tissue or plant cells comprising the introduction of a nucleic acid molecule, expression cassette or vector of the invention into a plant cell and, optionally, regenerating a transgenic plant or plant tissue therefrom. The transgenic plants expressing the fusion protein can be of use in monitoring the transport or movement of hormones throughout and between the organs of an organism, such as to or from the soil. The transgenic plants expressing transporters of the invention can be of use for investigating metabolic or transport processes of, e.g., organic compounds with a timely and spatial resolution.

Examples of species of plants that may be used for generating transgenic plants include but are not limited to monocotyledonous plants including seed and the progeny or propagules thereof, for example Lolium, Zea, Triticum, Sorghum, Triticale, Saccharum, Bromus, Oryzae, Avena, Hordeum, Secale and Setaria. Especially useful transgenic plants are maize, wheat, barley plants and seed thereof. Dicotyledenous plants are also within the scope of the present invention include but are not limited to the species Fabaceae, Solanum, Brassicaceae, especially potatoes, beans, cabbages, forest trees, roses, clematis, oilseed rape, sunflower, chrysanthemum, poinsettia and antirrhinum (snapdragon). The plant may be crops, such as a food crops, feed crops or biofuels crops. Exemplary important crops may include soybean, cotton, rice, millet, sorghum, sugarcane, sugar beet, tomato, grapevine, citrus (orange, lemon, grapefruit, etc), lettuce, alfalfa, fava bean and strawberries, rapeseed, cassava, miscanthus and switchgrass to name a few.

Methods for the introduction of foreign nucleic acid molecules into plants are well-known in the art. For example, plant transformation may be carried out using Agrobacterium-mediated gene transfer, microinjection, electroporation or biolistic methods as it is, e.g., described in Potrykus and Spangenberg (Eds.), Gene Transfer to Plants. Springer Verlag, Berlin, N.Y., 1995. Therein, and in numerous other references, useful plant transformation vectors, selection methods for transformed cells and tissue as well as regeneration techniques are described which are known to the person skilled in the art and may be applied for the purposes of the present invention.

In another aspect, the invention provides harvestable parts and methods to propagation material of the transgenic plants according to the invention which contain transgenic plant cells as described above. Harvestable parts can be in principle any useful part of a plant, for example, leaves, stems, fruit, seeds, roots etc. Propagation material includes, for example, seeds, fruits, cuttings, seedlings, tubers, rootstocks etc.

The present invention also provides methods of producing any of the fusion proteins of the present invention, the method comprising culturing a host cell in conditions that promote protein expression and recovering the fusion protein from the culture, wherein the host cell comprises a vector encoding a fusion protein, wherein the fusion protein comprises at least a first and second fluorescent protein, wherein the first and second fluorescent proteins emit wavelengths of light that are different from one another at least one plant hormone binding domain comprising an N-terminus and a C-terminus, wherein the binding domain changes three-dimensional conformation upon specifically binding to a plant hormone, and at least a first and second fluorescent protein linker peptide, wherein the first fluorescent protein linker peptide links the first fluorescent protein to the N-terminus of the at least one plant hormone binding domain and the second fluorescent protein linker peptide links the second fluorescent protein to the C-terminus of the at least one plant hormone binding domain.

The protein production methods generally comprise culturing the host cells of the invention under conditions such that the fusion protein is expressed, and recovering said protein. The culture conditions required to express the proteins of the current invention are dependent upon the host cells that are harboring the polynucleotides of the current invention. The culture conditions for each cell type are well-known in the art and can be easily optimized, if necessary. For example, a nucleic acid encoding a fusion protein of the invention, or a construct comprising such nucleic acid, can be introduced into a suitable host cell by a method appropriate to the host cell selected, e.g., transformation, transfection, electroporation, infection, such that the nucleic acid is operably linked to one or more expression control elements as described herein. Host cells can be maintained under conditions suitable for expression in vitro or in vivo, whereby the encoded fusion protein is produced. For example host cells may be maintained in the presence of an inducer, suitable media supplemented with appropriate salts, growth factors, antibiotic, nutritional supplements, etc., which may facilitate protein expression. In additional embodiments, the fusion proteins of the invention can be produced by in vitro translation of a nucleic acid that encodes the fusion protein, by chemical synthesis or by any other suitable method. If desired, the fusion protein can be isolated from the host cell or other environment in which the protein is produced or secreted. It should therefore be appreciated that the methods of producing the fusion proteins encompass expression of the polypeptides in a host cell of a transgenic plant. See U.S. Pat. Nos. 6,013,857, 5,990385, and 5,994,616.

The invention also provides for methods of measuring plant hormones in a sample, comprising contacting the sample with a fusion protein of the present invention and subsequently measuring the fluorescent resonance energy transfer (FRET) that occurs between the first and second fluorescent proteins. Accordingly, the fusion proteins can be used in sensors for measuring target analytes in a sample, with the sensors comprising the fusion proteins of the present invention.

The fusion proteins of the current invention can be used to assess or measure the concentrations of more than one target analytes, i.e., plant hormone. As used herein, concentration is used as it is in the art. The concentration may be expressed as a qualitative value, or more likely as a quantitative value. As used herein, the quantification of the analytes can be a relative or absolute quantity. Of course, the quantity (concentration) of any of the analytes may be equal to zero, indicating the absence of the particular analyte sought. The quantity may simply be the measured signal, e.g., fluorescence, without any additional measurements or manipulations. Alternatively, the quantity may be expressed as a difference, percentage or ratio of the measured value of the particular analyte to a measured value of another compound including, but not limited to, a standard or another analyte. The difference may be negative, indicating a decrease in the amount of measured analyte(s). The quantities may also be expressed as a difference or ratio of the analyte(s) to itself, measured at a different point in time. The quantities of analytes may be determined directly from a generated signal, or the generated signal may be used in an algorithm, with the algorithm designed to correlate the value of the generated signals to the quantity of analyte(s) in the sample.

The fusion proteins of the current invention are designed to possess capabilities of continuously measuring the concentrations an analyte. As used herein, the term “continuously,” in conjunction with the measuring of an analyte, is used to mean the fusion protein either generates or is capable of generating a detectable signal at any time during the life span of the fusion protein. The detectable signal may be constant in that the fusion protein is always generating a signal, even if the signal is not detected. Alternatively, the fusion protein may be used episodically, such that a detectable signal may be generated, and detected, at any desired time.

The target analytes can be any plant hormone where the concentration is desired to be measured. For example, the target analytes may be abscisic acid (ABA) or a derivative thereof, an auxin (IAA), a gibberellin (GA) or jasmonic acid (JA) or a derivative thereof. In one embodiment, the target analytes are not labeled. While not a requirement of the present invention, the fusion proteins are particularly useful in an in vivo setting for measuring target analytes as they occur or appear in a plant or plant tissue. As such, the target analytes need not be labeled. Of course, unlabeled target analytes may also be measured in an in vitro or in situ setting as well. In another embodiment, the target analytes may be labeled. Labeled target analytes can be measured in an in vivo, in vitro or in situ setting.

Purified biosensor can also be incorporated into kits for measurement of ABA or hormone concentrations in various samples. The samples would require minimal processing, thus the kit would allow high-throughput ABA or other hormone measurement in complex samples using an appropriate plate fluorometer (e.g. TECAN M1000). This type of analysis can be used to measure the ABA content or other hormone content in different tissues, different individual plants or different populations of, for example, crop plants experiencing drought. Purification of bulk amounts of biosensor can be achieved after expression in Pichia pastoris, using pPinkFLIP vectors and a protease deficient strain of Pichia.

The examples herein are provided for illustrative purposed and are not intended to limit the scope of the invention in any way.

### EXAMPLES

### Example 1

### Cloning, Expression and Screening of Signaling Molecule Sensors

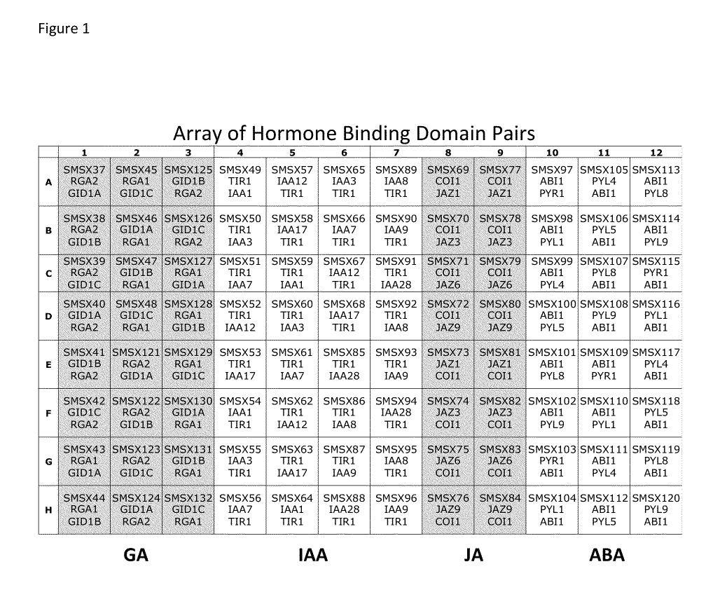

PCR amplification was used to screen for putative hormone binding domain coding sequences. Hormone binding domains were designed based on the function of endogenous hormone receptors and co-receptors from Arabidopsis thaliana. In cases where a designed hormone binding domain contained more than one sub-domain, overlap PCR was employed to obtain the full-length products. A novel sequence was designed to be a generic overlap site, i.e., X1 site, and the site was incorporated into the relevant primers. The X1 site codes for a short, flexible linker of ten amino acids (L10), but the site also serves as an acceptor for additional DNA sequences that code for linkers of differing sizes and molecular properties, i.e., a molecular spring of 45 amino acids (L45), a longer flexible linker of 60 amino acids (L60), an alpha-helix of 64 amino acids (L64), and the L45 and L64 combined (L111). In all cases, GATEWAY™ attB sites were incorporated into the final 5′ and 3′ primers so that the products could be recombined into the GATEWAY™ DONR vector pDONR221 using the GATEWAY™ BP reaction. The resulting library of GATEWAY™ Entry clones (FIG. 1) was then ready for recombination into GATEWAY™ destination vectors. A library of such GATEWAY™ destination vectors was created that would allow for the expression of the hormone binding domain flanked by two fluorescent proteins of a FRET pair, which constitutes the complete potential biosensor (Table IV). The library of Entry clones and library of destination vectors enabled a combinatorial, and thus high-throughput, approach to generating biosensor clones.

TABLE IV
Library of GATEWAY ™ destination vectors for multiple expression systems

| | | First | Second | | pFLIPi for | pFLIPi for | pDRFLIP | pZPuFLIP |
|---|---|---|---|---|---|---|---|---|
| | N- | fluorescent | fluorescent | C- | E. coli | in vitro | for yeast | for plant |
| Design # | tag | protein | protein | tag | expression | expression | expression | expression |
| 13 | 6H | CFPt10 | fIVFP | — | Yes | Yes | | |
| 14 | 6H | CFPt10 | t6VFP | — | Yes | Yes | | |
| 15 | 6H | CFPt10 | fIVFP | cMyc | Yes | Yes | | |
| 16 | 6H | CFPt10 | t6VFP | cMyc | Yes | Yes | | |
| 17 | — | CFPt8 | fIVFP | cMyc | Yes | Yes | | |
| 18 | — | CFPt8 | t6VFP | cMyc | Yes | Yes | | |
| 19 | — | CFPt10 | fIVFP | cMyc | Yes | Yes | | |
| 20 | — | CFPt10 | t6VFP | cMyc | Yes | Yes | | |
| 21 | — | TFP | fIVFP | cMyc | Yes | Yes | | |
| 22 | — | TFP | t6VFP | cMyc | Yes | Yes | | |
| 23 | — | TFPt9 | fIVFP | cMyc | Yes | Yes | | |
| 24 | — | TFPt9 | t6VFP | cMyc | Yes | Yes | | |
| 25 | 6H | TFP | VFP | — | Yes | | | |
| 26 | 6H | CFP | VFP | — | Yes | | | |
| 27 | 6H | TFP | AFPt9 | cMyc | Yes | | | |
| [symbol] | [symbol] | [symbol] | [symbol] | [symbol] | [symbol] | [symbol] | | |
| 29 | 6H | AFPt9 | TFP | — | Yes | | | |
| 30 | 6H | AFPt9 | mCer | cMyc | Yes | | Yes | Yes |
| 31 | 6H | AFPt9 | sCer | cMyc | Yes | | | |
| 32 | 6H | AFPt9 | t7CFPt9 | cMyc | Yes | | Yes | Yes |
| 33 | 6H | AFPt9 | t7sCFPt9 | cMyc | Yes | | | |
| 34 | 6H | AFPt9 | t7TFPt9 | cMyc | Yes | | Yes | Yes |
| 35 | 6H | AFPt9 | TFPt9 | cMyc | Yes | | Yes | Yes |
| 36 | 6H | AFPt9 | Cer | cMyc | Yes | | Yes | Yes |
| 37 | 6H | Cit | Cer | cMyc | Yes | | Yes | Yes |
| 38 | 6H | sCit | sCer | cMyc | Yes | | Yes | Yes |
| 39 | 6H | sAFPt9 | t7sCFPt9 | cMyc | Yes | | Yes | Yes |
| 40 | 6H | Cit | TFPt9 | cMyc | Yes | | | |
| 41 | 6H | Cit | t7TFPt9 | cMyc | Yes | | | |
| 42 | 6H | Cit | mCer | cMyc | Yes | | Yes | Yes |
| 43 | 6H | sAFPt9 | sCer | cMyc | Yes | | Yes | Yes |
| 44 | 6H | tdTom | mAme1.2 | cMyc | Yes | | | |
| 45 | 6H | tdTom | AcGFP | cMyc | Yes | | | |
| 46 | 6H | AcGFP | tdTom | cMyc | Yes | | | |
| 47 | 6H | mAme1.2 | tdTom | cMyc | Yes | | | |
| 48 | 6H | AFPt9 | mTrq2 | cMyc | Yes | | Yes | Yes |
| 49 | 6H | AFPt9 | t7mTrq2t9 | cMyc | Yes | | Yes | Yes |
| 50 | 6H | sAFPt9 | t7sTrq2t9 | cMyc | Yes | | Yes | Yes |
| 51 | 6H | sAFPt9 | sTrq2 | cMyc | Yes | | Yes | Yes |
| 52 | 6H | x | mTrq2 | cMyc | Yes | | Yes | |
| 53 | 6H | AFPt9 | mAme1.2 | cMyc | Yes | | | |
| 54 | 6H | AFPt9 | AcGFP | cMyc | Yes | | | |
| 55 | 6H | AFPt9 | tdTom | cMyc | Yes | | | |
| 56 | 6H | x | sTrq2 | cMyc | Yes | | Yes | |
| 57 | 6H | x | t7sTrq2t9 | cMyc | Yes | | Yes | |

| | | |
|---|---|---|
| Abbreviation | Full name | Notes |
| VFP | Venus | Yellow |
| AFP | Aphrodite | Yellow (codon changed Venus) |
| ChFP | mCherry | Red |
| TFP | mTeal | Blue |
| CFP | eCyan | Blue |
| Cit | Citrine | Yellow |
| Cer | Cerulean | Blue |
| AcGFP | Green | Green |
| Tom | Tomato | Orange/red |
| Ame | Ametrine | Green/yellow |
| Trq | Turquoise | Blue |
| td | tandem dimer | brighter variant |
| s | sticky | dimer tendency variant |
| m | monomeric | dimer tendency variant |
| t# | truncation | N- or C-terminal |
| w/out s or m | weak dimer | original eGFP |
| x | no fluorophore | useful for intramolecular SMS |

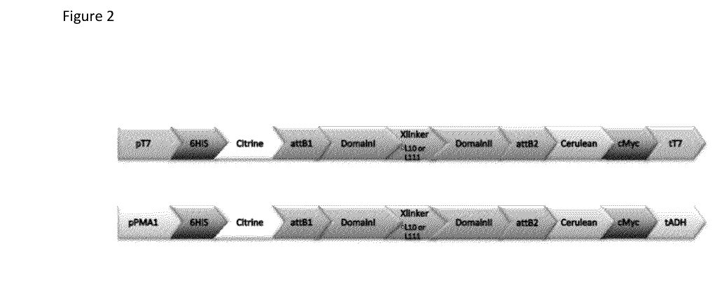

Expression and screening hormone biosensors using Saccharomyces cerevisiae as a eukaryotic host cell to better express the eukaryotic hormone binding domains. S. cerevisiae can be adapted to a high-throughput expression system. A new library of GATEWAY™ destination vectors was created for expression in yeast (Table IV, FIG. 2). Although expression of SMS in S. cerevisiae was successful as determined by fluorescence emissions, purification of sensors from the host cells was not possible because of cleavage of the hormone binding domains of the SMS proteins upon cell lysis, which can be important because the rate of transport of many plant hormones into yeast cells is low. It was subsequently found that full-length SMS proteins could be found in and purified from the cell lysate of protease deficient strains of yeast. SMS proteins were subsequently screened from cell lysates and after purification using metal-affinity chromatography, if the clones included an N-terminal 6HIS tag.

Subsequent experiments identified protocols for yeast cell transformation, culture and lysis, metal-affinity chromatography, and fluorescence analysis after hormone treatment in a 96-well format. Thus, a novel high-throughput platform for creation and screening of SMS had been developed.

### Example 2

### Abscisic Acid (ABA) Sensor

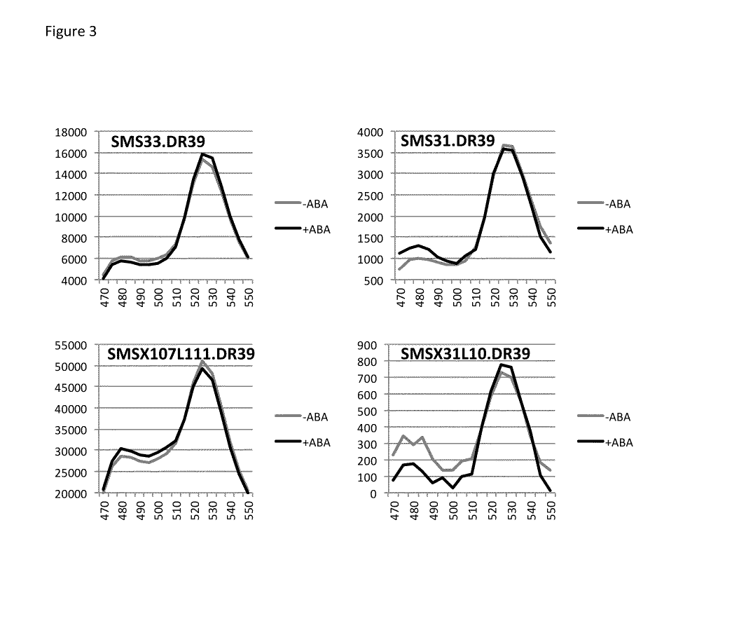

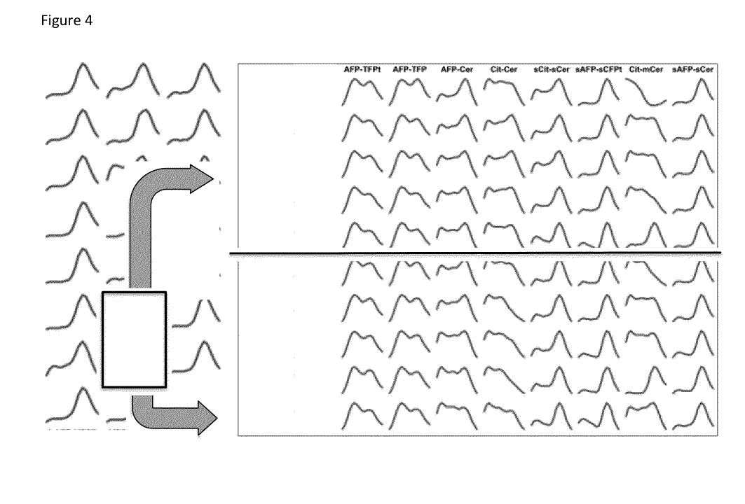

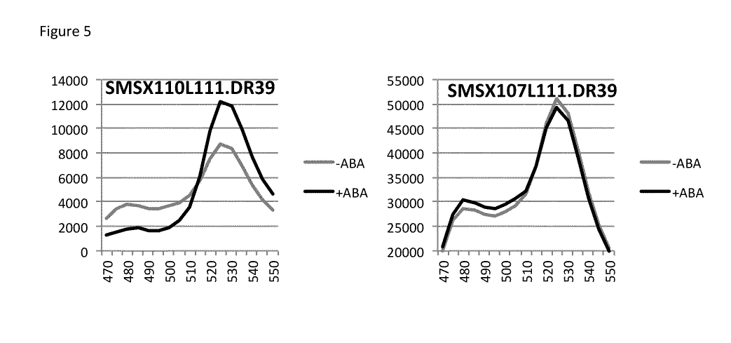

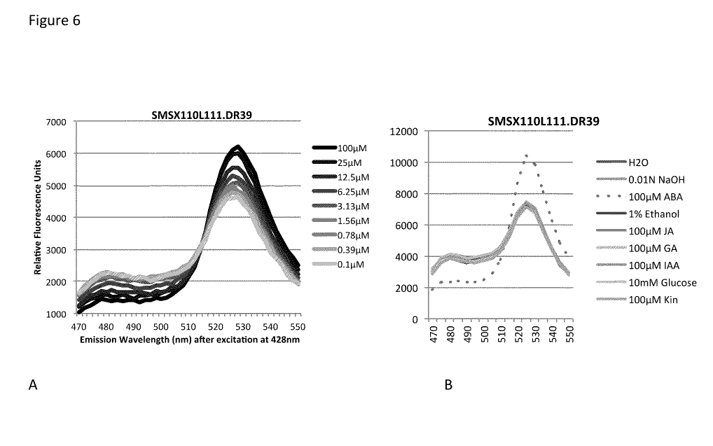

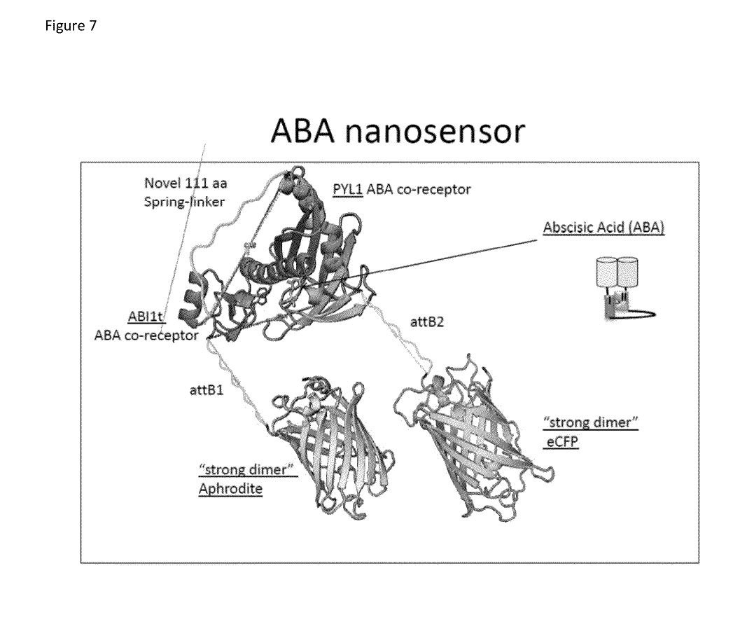

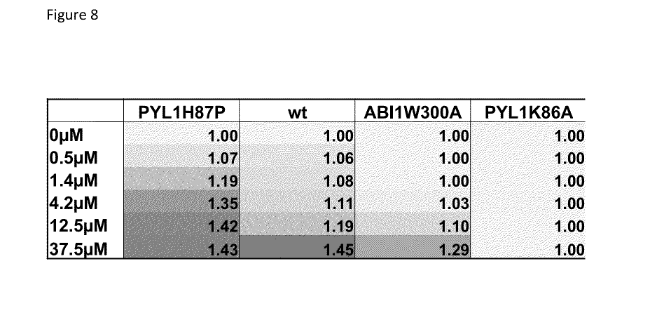

Screening of SMS proteins for hormone responses resulted in the identification of a series of abscisic acid (ABA) biosensors (FIG. 3). ABA responsive biosensors were found with both single domain designs and designs with multiple subdomains. ABA biosensors can also be derived from multiple members of the PYL and PP2C domain sets, with either subdomain being the N-terminal domain, and with multiple variants of linkers connecting subdomains (FIG. 3). The combinatorial SMS screen was used to optimize the subdomain combination, subdomain ordering, fluorescent protein FRET pair and FRET ratio change of the ABA biosensors (FIGS. 4 and 5). The ABA biosensor SMSX110L111.DR39 can be used to specifically detect ABA concentration in in vitro samples ranging from ˜1-100 μM (FIG. 6). The sensor behavior was similar to the behavior of prior experiments using the ABA receptor complexes (FIGS. 7 and 8). For example, several crystal structures of ABA receptor and PP2C co-receptor complexes can serve as a guide for mutations that may be able to diminish or disrupt ABA binding. Other results indicated that mutations that reduce the homo-dimerization tendencies of particular ABA receptors (including PYR1 and PYL1) result in higher affinity ABA binding. The ABA biosensor SMSX110L45.DR38 was used as the basis to generate mutant sensors with higher, lower and no ABA affinity (FIG. 8). This set of sensors expands the range of ABA concentrations that can be detected with ABA biosensors in vivo and in vitro.

### Example 3

### Gibberellin (GA) Sensor

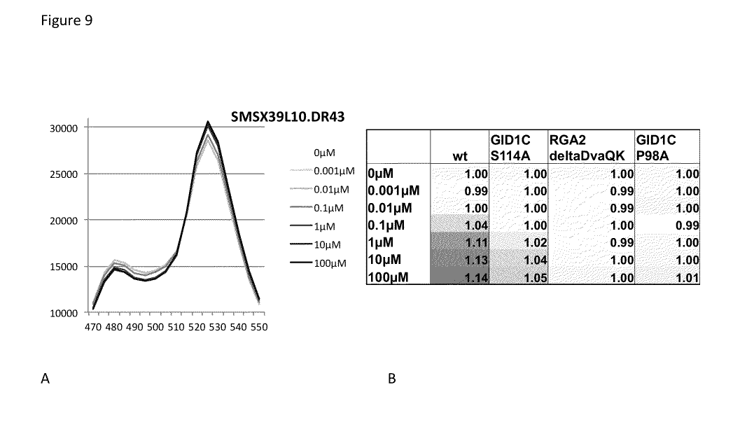

The same methodology used to identify the ABA biosensors was also applied to identify and optimize GA biosensors. The GA biosensor SMSX39L10.DR43 (RGA2 and GID1C sub-domains, L10 linker, Venus and Cerulean fluorescent proteins) behaves similarly to GA binding domains (FIG. 9). The GA biosensors can detect GA in samples with high-affinity (FIG. 9).

### Example 4

### Jasmonate (JA) and Auxin Sensor

The same methodology used to identify and prepare the ABA and GA biosensors is used to identify and optimize biosensors that detect jasmonate (JA) and auxin (IAA). The JA and IAA biosensors would include at least one binding domain, a linker, such as L10 and a reporter such as Venus and Cerulean fluorescent proteins. The JA and IAA biosensors can detect JA and IAA in samples, respectively.

### Example 5

The mutant series of ABA and GA biosensors were been cloned with pZPuFLIP destination vectors. These vectors allow for expression of the biosensors in plant cells after Agrobacterium mediated plant transformation. The ABA and GA biosensors were expressed in Arabidopsis thaliana, but any transformable organism can express the biosensor for high-resolution analysis of ABA in vivo. The host transgenic plane comprising a biosensor, e.g., an ABA biosensor, allows for mapping of ABA distributions over time before, during and after a stress response. The biosensor also allows for studying and analyzing developmental processes or effect of mutations that can reveal patterns and dynamics heretofore undetectable.

What is claimed is:

1. A fusion protein comprising
a) at least a first and second fluorescent protein, wherein the first and second fluorescent proteins emit wavelengths of light that are different from one another
b) at least one plant hormone binding domain comprising an N-terminus and a C-terminus, wherein the binding domain changes three-dimensional conformation upon specifically binding to a plant hormone, and
c) at least a first and second fluorescent protein linker peptide, wherein the first fluorescent protein linker peptide links the first fluorescent protein to the N-terminus of the at least one plant hormone binding domain and the second fluorescent protein linker peptide links the second fluorescent protein to the C-terminus of the at least one plant hormone binding domain.

2. The fusion protein of claim 1, wherein the at least one plant hormone binding domain comprises at least two subdomains that are linked together with a subdomain linker peptide.

3. The fusion protein of claim 2, wherein the first and second fluorescent protein linker peptides are the same.

4. The fusion protein of claim 3, wherein the subdomain linker peptide is different from the first and second fluorescent protein linker peptides.

5. The fusion protein of claim 4, wherein the subdomain linker peptide comprises the amino acid sequence of SEQ ID NO:1 or SEQ ID NO:2.

6. The fusion protein of claim 5, wherein the first and second fluorescent protein linker peptides comprise an amino acid sequence of SEQ ID NO:3 or SEQ ID NO:4.

7. The fusion protein of claim 6, wherein the first and second fluorescent proteins are selected from the group consisting of green fluorescent protein (GFP), yellow fluorescent protein (YFP), cyan fluorescent protein (CFP), citrine, cerulean, VENUS and teal fluorescent protein (TFP).

8. The fusion protein of claim 7, wherein the plant hormone binding domain specifically binds to abscisic acid (ABA) or a derivative thereof, an auxin (IAA), a gibberellin (GA) and jasmonic acid (JA) or a derivative thereof.

9. The fusion protein of claim 8, wherein the plant hormone binding domain specifically binds GA and the plant hormone binding domain comprises two subdomains with the first subdomain selected from the group consisting of RGA1 and RGA2, and the second subdomain selected from the group consisting of GID1A, GID1B and GID1C.

10. The fusion protein of claim 8, wherein the plant hormone binding domain specifically binds IAA and the plant hormone binding domain comprises two subdomains with the first subdomain being TIR1, and the second subdomain selected from the group consisting of IAA1, IAA3, IAA7, IAA12, IAA17, IAA28, IAA18 and IAA9.

11. The fusion protein of claim 8, wherein the plant hormone binding domain specifically binds JA or a derivative thereof and the plant hormone binding domain comprises two subdomains with the first subdomain being COI1, and the second subdomain selected from the group consisting of JAZ1, JAZ3, JAZ6 and JAZ9.

12. The fusion protein of claim 8, wherein the plant hormone binding domain specifically binds ABA or a derivative thereof and the plant hormone binding domain comprises two subdomains with the first subdomain selected from the group consisting of ABI1 and HAB1, and the second subdomain selected from the group consisting of PYR1, PYL1, PYL4, PYL5, PYL8 and PYL9.

13. A nucleic acid that encodes the fusion protein of claim 1.

14. A vector comprising the nucleic acid of claim 13.

15. A host cell comprising the vector of claim 14.

16. A plant comprising the host cell of claim 15.

17. A method of producing a fusion protein, the method comprising culturing a host cell in conditions that promote protein expression and recovering the fusion protein from the culture, wherein the host cell comprises a vector encoding a fusion protein, wherein the fusion protein comprises
a) at least a first and second fluorescent protein, wherein the first and second fluorescent proteins emit wavelengths of light that are different from one another
b) at least one plant hormone binding domain comprising an N-terminus and a C-terminus, wherein the binding domain changes three-dimensional conformation upon specifically binding to a plant hormone, and
c) at least a first and second fluorescent protein linker peptide, wherein the first fluorescent protein linker peptide links the first fluorescent protein to the N-terminus of the at least one plant hormone binding domain and the second fluorescent protein linker peptide links the second fluorescent protein to the C-terminus of the at least one plant hormone binding domain.

18. A method of detecting a plant hormone in a sample, the method comprising contacting the fusion protein of claim 1 with the sample and determining the amount of fluorescence resonance energy transfer (FRET) between the first and second fluorescent proteins that occurs after the plant hormone binds to the plant hormone binding moiety of the fusion protein.

19. The method of claim 18, wherein the plant hormone is selected from the group consisting of abscisic acid (ABA) or a derivative thereof, an auxin (IAA), a gibberellin (GA) and jasmonic acid (JA) or a derivative thereof.

20. The method of claim 19, wherein the sample is in a plant or tissue thereof.


SEQUENCE LISTING (7 sequences)

SEQ ID NO: 1 - protein, 12 aa, Artificial Sequence
  Linker Domain L10
ASGAPGAGRSSG

SEQ ID NO: 2 - protein, 52 aa, Artificial Sequence
  Linker Domain L45
ASGAPGPGGAGPGGAGPGGAGPGGAGPGGAGPGGAGPGGAGPGGAGAGRSSG

SEQ ID NO: 3 - protein, 65 aa, Artificial Sequence
  Linker Domain L60
ASGAPGGSGGSGGSGGSGGSGGSGGSGGSGGSGGSGGSGGSGGCGGSGGSGGSGGSGGSAGRSSG

SEQ ID NO: 4 - protein, 71 aa, Artificial Sequence
  Linker Domain L64
ASGASMALEKMALEKMALEKGSMALEKMALEKMALEKMALEKMALEKGSMALEKMALEKMALEKPSGRSSG

SEQ ID NO: 5 - protein, 118 aa, Artificial Sequence
  Linker Domain L111
ASGAPGPGGAGPGGAGPGGAGPGGAGPGGAGPGGAGPGGAGPGGAGAGRNASMALEKMALEKMALEKGSMALEKMALEKMALEKMALEKMALEKGSMALEKMALEKMALEKPSGRSSG

SEQ ID NO: 6 - protein, 11 aa, Artificial Sequence
  Linker Domain B1
DITSLYKKAGF

SEQ ID NO: 7 - protein, 11 aa, Artificial Sequence
  Linker Domain B2
HPAFLYKVVIS